(19) United States
(12) Patent Application Publication
(10) Pub. No.: US 2007/0102003 A1
(43) Pub. Date: May 10, 2007

(54) Extracting tool for cervical diaphragm

(75) Inventor: David P. Newman, Fort Morgan, CO (US)

Correspondence Address: Joseph G. Nauman, 696 Renolda Woods Ct., Dayton, OH, 45429-3415, US

(73) Assignee: CAMAX Tool Company, Inc.

(21) Appl. No.: 11/270,141

(22) Filed: Nov. 9, 2005

(51) Int. Cl. A61F 6/06

(52) U.S. Cl. 128/837; 128/834

(57) ABSTRACT

A small relatively rigid tool, without sharp or pointed features, is disclosed, which can easily be inserted between the cervical tissue and the rim of an in-place diaphragm (or cervical cap), sometimes referred to as a pessary device or appliance. The tool has an elongated substantially rigid body with a blunt domed head at one end of the body, the body being manipulated to engage the head with and around a small portion of the rim of the diaphragm can be used to engage such rim and withdraw it. The so-engaged tool can then be manipulated to pull the diaphragm away from the cervix and out through the vaginal passage with minimal force. The extracting tool is also useful (with minor modifications in its form) in the case of removing a cervical cap. Also disclosed is the method of using such tool in the removal of a pessary appliance.

### FIELD OF THE INVENTION

The present invention relates to a simple manually manipulative tool for removing an in-place cervical diaphragm, namely a so-called conception preventing device which, when properly installed, prevents the passage of sperm past the cervical opening into the female uterus.

### BACKGROUND OF THE INVENTION

FIGS. 1 and 2 of the drawings, and the following related description, are derived from the John's Hopkins Family Health Book, pages 1118 & 1161, © 1999 by the John Hopkins University. “A diaphragm is a round, dome-shaped piece of rubber encircled with a thick reinforced, flexible rubber rim. It is designed to cover the cervix and prevent sperm from entering. A cervical cap is also designed to cover the cervix and prevent the entry of sperm. It is hard plastic and thimble-shaped, and usually not the choice of younger women. Both must be used with spermacidal cream or foam, and left in place for 6 hours after intercourse in the case of the diaphragm (to ensure that the spermacide has killed all the sperm), or for 48 hours in the case of the cervical cap. A woman must be fitted for a cervical cap or diaphragm by her doctor or a local family planning clinic.” FIG. 2 depicts the placement of a diaphragm D in its functioning position.

Present methods employed for diaphragm removal all require the manipulation of fingers and finger tips in somewhat awkward hand and arm positions. Complicating removal is the smooth featureless nature of the diaphragm and its slippery surface. After being in place for the recommended period of time, a suction-like attachment often develops, further aggravating and complicating removal efforts. The compliant pressing between the rim and the tissue against which it is placed must be carefully overcome in order to engage the rim and withdraw the diaphragm.

For some women, these conditions are sufficient impediments that use of a diaphragm is highly annoying, even though its benefits as a temporary and generally non-chemical mechanical barrier may be for them considered superior to other methods. What is needed is an apparatus and method to relieve the difficulties of diaphragm removal that is simple, easy to use, comfortable, and manufacturable easily and at low cost.

### SUMMARY OF THE INVENTION

The present invention provides a small relatively rigid tool, without sharp or pointed features, which can easily be inserted between the cervical tissue and the rim of an in-place diaphragm (or cervical cap), sometimes referred to as a pessary device or appliance, and which can be used to engage around a small portion of such rim and withdraw it and the remainder of the diaphragm away from the cervix and out through the vaginal passage with minimal force. The extracting tool is also useful (with minor modifications in its form) in the case of removing a cervical cap. The invention also encompasses the method of using such tool in the removal of a pessary appliance.

Further objects and advantages of this invention will be apparent from the following description and the drawings.

### BRIEF DESCRIPTION OF THE DRAWINGS

FIG. 1 is a diagram (Prior Art) of the human female reproductive organs including the vagina, the uterus with its cervical opening at the inner end of the vaginal passage, and adjacent organs in the lower abdomen;

FIG. 2 is an enlarged diagram (Prior Art) of the cervix with a diaphram D in place;

FIG. 3 is a side view of the extracting tool provided by the invention;

FIG. 4 is a diagram illustrating the manipulation of the tool by the fingers of the user; and

FIG. 5 illustrates a position of entry of the tool head past the rim of an in-place diaphragm, hooking around its rim.

### DESCRIPTION OF THE PREFERRED EMBODIMENT

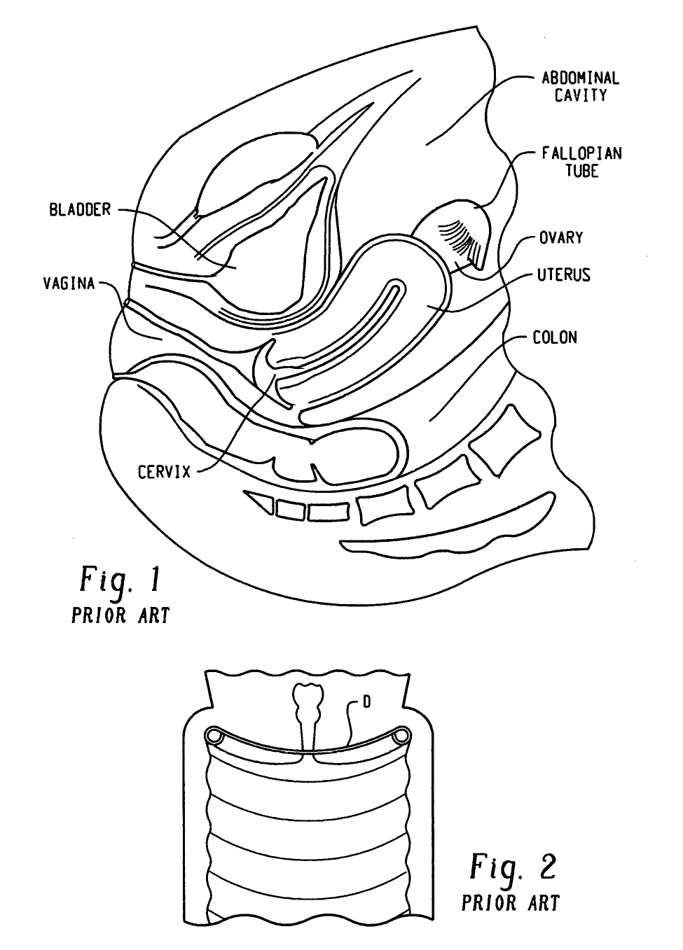

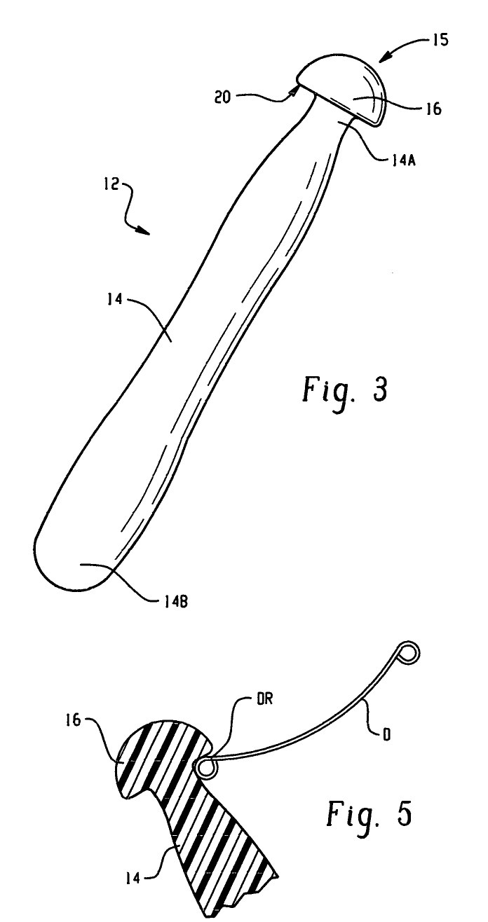

FIGS. 1 and 2, as mentioned in the Background, illustrate the placement of a cervical diaphragm D with a rim Dr across the cervix, wherein it is placed prior to intercourse, and is recommended to be kept in place during and subsequent to intercourse for a period of time in the order of six hours. FIG. 3 illustrates the extracting tool 12, which is preferably machined or molded of a relatively rigid synthetic material, e.g. a polycarbonate such as Lexan, or from Nylon, Urethane, or the like. Prefereably, the material should be susceptible to sterilization procedures.

The tool comprises an exteriorly smooth generally cylindrical body and handle 14 having at one end 14A a blunt head 15 configured into the shape of an umbrella or mushroom with a domed end surface 16 terminated at its joint to body 14 as a smooth rounded generally circular edge which merges toward the end 14A of body 14. This edge defines a lip 20 with a smooth undersurface 21 facing toward the other end 14B of the body, and extending generally radially from the body at the underside of head 15. The preferred embodiment presents a continuous lip 20, but it within the scope of this invention for such lip to extend for less than 360° so long as its edges are smoothly tapered (faired) into the remainder of the head 15 such that no abrupt edge or point is present along the undersurface 21.

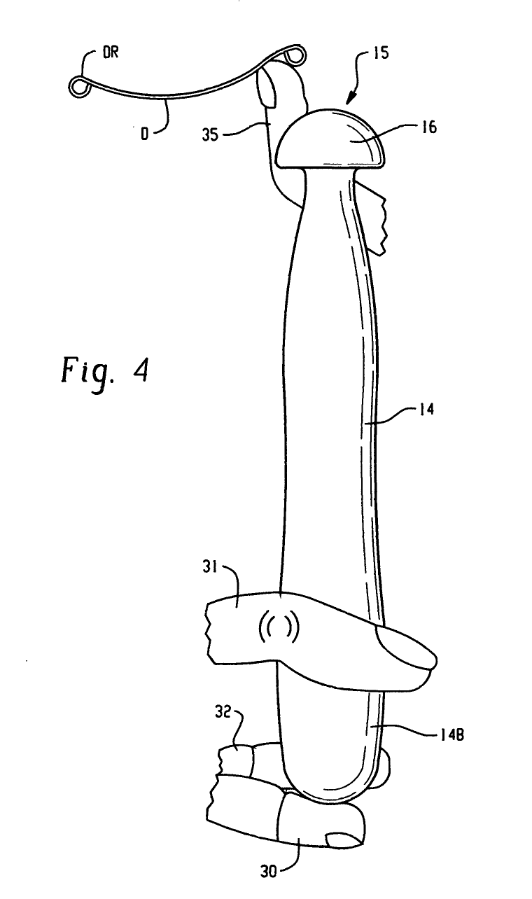

Regarding the method of using this novel tool, as shown in FIG. 4, the user can hold the tool with an index finger 30 of one hand supporting the end 14B of the handle or main body 14. The sides of the handle can be steadied by the thumb 31 and second finger 32 of that hand. The index finger 35 of the other hand is used to locate the rim Dr and the head 15 is moved, as by sliding it along finger 35, to the outer edge of the rim where it is pressing against cervical tissue. The tool is then pressed past the rim Dr until the lip 20 locates behind the adjacent portion of rim Dr (FIG. 5) and the diaphragm D can be released by drawing the tool outward away from the cervix. It should be noted (as previously stated) that head 15 is smooth all around its periphery and does not present even the smallest surface feature which might irritate the sensitive tissue which it will contact during use. The tool head can be moved along and around (on the inward side of) rim Dr as part of assisting in dislodging the diaphragm if the user senses resistance to removal. The tool can then be used to remove, or assist the fingers in removing, the diaphragm.

Alternative geometry of the tool may be employed without departing from the scope of this invention. For example, the cylindrical characteristic of the handle and head may be flattened, e.g. into an elliptical type cross-section, and the lip may extend only part way around the circumference of the tool. Such geometrical alternatives can enhance performance of the tool when removing, for example, a cervical cap.

While the forms of device herein described, and the method of its use, constitute preferred embodiments of this invention, it is to be understood that the invention is not limited to these precise forms and method of use, and that changes may be made therein without departing from the scope of the invention which is defined in the appended claims.

What is claimed is:

1. A device for assisting in the removal of a pessary appliance, comprising
a generally cylindrical elongated and substantially rigid body which provides a manipulating handle,
a blunt domed head on one end of said body extending at least partially around and outward therefrom in a sideways direction to provide a lip,
said head being shaped to slip between a part of the rim portion of the pessary device and surrounding tissue upon insertion into a vaginal passage and its cervical cavity, and to capture a portion of such rim with said lip and then assist in the removal of the pessary appliance.

2. A device as defined in claim 1, wherein said head is formed with a rounded exterior surface.

3. A device as defined in claim 1, wherein said body and head are integral parts of an integral tool device, and the exterior of said tool device is smooth and free from rough and sharp surface features.

4. A device as defined in claim 1, wherein said tool device is formed from a relatively inert polymer.

5. A device as defined in claim 1, wherein said head is in the form of a semi-spherical protruberance at one end of said body.

6. A method of removing an in-place pessary appliance having a rim pressing against tissue at the cervix using a tool as defined in claim 1, comprising the steps of
a) inserting the tool into proximity with the appliance rim,
b) moving the lip of the tool between the rim and the surrounding tissue and hooking the lip partially about the rim,
c) moving the lip at least partially about the rim to release the rim of the device from the tissue, and
d) then pulling the tool outward to assist in removal of the pessary device.
